(19) United States
(12) Patent Application Publication
(10) Pub. No.: US 2012/0171077 A1
(43) Pub. Date: Jul. 5, 2012

(54) System and method for decontamination

(75) Inventors: John H. Bridges, III, Stafford, VA (US); Dennis M. Baca, Gainesville, VA (US)

(73) Assignee: UNITED STATES POSTAL SERVICE

(21) Appl. No.: 11/976,089

(22) Filed: Oct. 19, 2007

Related U.S. Application Data
(63) Continuation of application No. 10/882,214, filed on Jul. 2, 2004, now abandoned.
Application No. 10/882,214 is a continuation-in-part of application No. 10/684,635, filed on Oct. 14, 2003, now abandoned.
Application No. 10/684,635 is a continuation-in-part of application No. 10/422,708, filed on Apr. 24, 2003, now Pat. No. 7,459,700.
(60) Provisional application No. 60/484,924, filed on Jul. 3, 2003.
(60) Provisional application No. 60/374,813, filed on Apr. 24, 2002.

(51) Int. Cl. A61L 2/16; A61L 2/20

(52) U.S. Cl. 422/33

(57) ABSTRACT

Systems and methods for decontaminating a facility, for example, in response to a biological attack. An exclusion zone, a support zone, and a contamination reduction zone may be established. The exclusion zone may have a suspected contaminant in one or more of its areas. The contamination reduction zone may be between the exclusion zone and the support zone. The exclusion zone may have an agent introduced into a pressurized environment within the exclusion zone to substantially eliminate the contaminant. A sample may be taken to determine the presence of the contaminant.

### CROSS-REFERENCE TO RELATED APPLICATIONS

This non-provisional application is related to and claims priority of Provisional Application No. 60/484,924 filed on Jul. 3, 2003, in the names of John BRIDGES III and Dennis BACA, and titled ANTHRAX REMEDIATION METHOD, the contents of which are fully incorporated herein by reference. This non-provisional application is a Continuation-In-Part of application Ser. No. 10/684,635 filed on Oct. 14, 2003, in the names of John BRIDGES III and Dennis BACA, and titled ANTHRAX REMEDIATION AND RESPONSE, the contents of which are fully incorporated herein by reference. This non-provisional application is also a Continuation-In-Part of application Ser. No. 10/422,708 filed on Apr. 24, 2003, in the names of John BRIDGES III and Dennis BACA, and titled ANTHRAX REMEDIATION AND RESPONSE, the contents of which are fully incorporated herein by reference. Application Ser. No. 10/422,708 claims priority to Provisional Application No. 60/374,813, filed on Apr. 24, 2002, the contents of which are fully incorporated herein by reference.

### DESCRIPTION OF THE INVENTION

1. Field of the Invention

This invention relates generally to methods for responding to bio-terrorism attacks and, more specifically, relates to a method or methods for responding to a bio-terrorism attack that addresses the decontamination of goods, vehicles, facilities, and personal protection equipment that are at least potentially contaminated as a result of such an attack and/or its remediation. Preferably, these individual methods are integrated into a coordinated response, or battle plan, following a bio-terrorism attack.

2. Background of the Invention

In 2001, the world witnessed the danger posed by a bio-terrorism attack. Beginning in the fall of 2001, a series of letters containing spores from the bacterium Bacillus anthracis were sent through the U.S. Postal Service. Handlers and recipients of certain of these letters contracted anthrax, with more than 20 becoming ill and five dying. The U.S. Postal Service was granted authority and responsibility for remediation of anthrax in U.S. Postal Service facilities and equipment by the U.S. Government by, for example, the National Oil and Hazardous Substances Pollution Contingency Plan, Part 300.

This incident posed a number of challenges to those tasked with the responsibility for remediation. These included; (a) devising a method for treating mail that had either been exposed to anthrax spores, or where there was at least a concern of potential exposure; (b) devising and demonstrating a method for decontaminating vehicles used to transport such mail for decontamination or otherwise; (c) decontaminating a facility where there has been a bio-terrorism attack or at least the possibility of one; and (d) devising a decontamination method for persons (including specifically their protective gear) who are required to enter a facility where there has been a possible bio-terrorism attack.

These methods address the need or effective responses to bio-terrorism. They have application not only to mail items, vehicles and facilities, but to other targets of bio-terrorism. Further, they have application to anthrax-type bio-terrorism attacks, as well as attacks utilizing other biological agents.

### SUMMARY OF THE INVENTION

In accordance with one aspect of the invention, a method for decontaminating a facility is provided. The method includes creating a pressurized environment inside the facility having a suspected contaminant in one or more areas; introducing an agent to the pressurized environment to substantially eliminate the contaminant; and sampling at least one area to determine a presence of the contaminant in a sample.

There is also provided a system for decontaminating a facility. The system includes an exclusion zone, wherein the exclusion zone includes some or all of the facility. The system also includes a contamination reduction zone located adjacent to the exclusion zone. Further, the system includes a support zone located at entry points to the contamination reduction zone.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

The accompanying drawings, which are incorporated in and constitute a part of this specification, illustrate one (several) embodiment(s) of the invention and together with the description, serve to explain the principles of the invention.

### BRIEF DESCRIPTION OF THE DRAWINGS

FIG. 1 is a flow chart illustrating the treatment of mail items suspected of exposure to a biological agent.

FIG. 2 is a flow chart illustrating the remediation of a vehicle interior possibly exposed to a biological agent.

FIG. 3 is a block diagram illustrating the relative position of the exclusion zone, the contamination reduction zone, and the support zone relative to one another.

FIG. 4 is a flow chart illustrating the steps followed by a person seeking to enter an exclusion zone, exit therefrom, and decontaminate his or her personal protective equipment.

### DESCRIPTION OF THE EMBODIMENTS

A bio-terrorism attack poses a number of challenges for a proper and comprehensive response. While at least certain of the methods disclosed herein have been utilized in connection with a response to an anthrax-type of attack, it should be noted that they could be utilized with other biological agents. In this regard, the following is a classification of contaminants or biological agents which may be used as bioweapons, and for which remediation as herein described is believed and now proven effective:

1. Bacteria—bacteria are small free-living organisms, most of which may be grown on solid or liquid culture media. They have a structure generally consisting of nuclear material disposed within cytoplasm, and are bounded by a cell membrane. Bacteria reproduce by simple division. The diseases that bacteria produce often respond to specific therapy with antibiotics.

Bacillus anthracis is an example of a bacteria type of biological agent. It was the first bacterium shown to be the cause of a disease. It most commonly occurs in wild and domestic lower vertebrates, such as cattle, sheep, goats, camels, antelopes, and other herbivores. It can also occur in humans, typically when they are exposed to infected animals or to tissue from infected animals. The disease is acquired by skin contact with the bacteria or by inhaling the bacteria spores. The spores are capable of attaching to the clothing of a person who has entered a contaminated area.

Anthrax infection can occur in three forms: cutaneous, inhalation, and gastrointestinal. Cutaneous anthrax occurs after the bacteria contact skin cuts or abrasions. Usually within two weeks, an itchy skin lesion develops that is similar to an insect bite. This lesion may later blister and then break down, resulting in a black, frequently painless, ulcer. In 20% of cases where the individual is untreated, the infection may spread through the bloodstream and become fatal. Otherwise, with treated individuals, death from cutaneous anthrax is extremely rare.

Inhalation anthrax develops when anthrax spores enter the lungs. Development of the actual disease occurs after the spores germinate, a process that may take up to 60 days. Once the spores germinate, several toxic substances are released. This results in hemorrhage, swelling, and tissue death. In the first stage of inhalation anthrax, the individual can experience fever, headache, cough, shortness of breath, and chest pain. The second stage develops suddenly, and is characterized by shortness of breath, fever, and shock. Most of these cases are fatal.

Gastrointestinal anthrax occurs with ingestion of contaminated meat. Disease in these cases usually develops within one week and can affect the upper portion of the gastrointestinal tract or the intestines and colon. The infection can enter the bloodstream and result in death.

2. Viruses—viruses are organisms that require living cells in which to replicate, and are therefore dependent upon the cells of the host that they infect. Their stability in the environment is very variable. They produce diseases which generally do not respond to antibiotics, but which may be responsive to anti-viral compounds. Supportive care (e.g., fluids, anti-inflammatories and rest) is often the only treatment for viral infections.

3. Rickettsiae—these are microorganisms that have characteristics common to both bacteria and viruses. Like bacteria, they possess metabolic enzymes and cell membranes, utilize oxygen and are susceptible to broad-spectrum antibiotics. They resemble viruses, on the other hand, in that they grow only within living cells.

4. Chlamydia—these are obligatory intracellular parasites that are incapable of generating their own energy source. Like bacteria, they are susceptible to broad-spectrum antibiotics. Like viruses, they require living cells for multiplication.

5. Fungi—these are primitive plants that utilize photosynthesis, are capable of anaerobic growth, and that draw nutrition from decaying vegetable matter. Most fungi form spores. Free-living forms are found in the soil. Fungal diseases may respond to various anti-microbial drugs.

6. Toxins—these are poisons that are produced by organisms. Toxins do not grow or reproduce. They are more easily controlled than live organisms. The potency of most toxins are such that very small doses will cause illness and/or death.

Evidence of a biological attack utilizing a biological agent can come in a number of ways. It is possible that the actual bioterror delivery system (e.g., the release of powdered material from processed mail or packages) may be observed. It can also come from the observance of any of the following:

1. Unusual number of casualties—a large number of casualties within a 48 to 72 hour period suggests that there may have been an attack with a microorganism. If the casualties occur within minutes to an hour of each other, a toxin is a more likely suspect. A large number of clinical cases relative to the number of exposed individuals, or other epidemiological evidence of a massive single source disease outbreak may all be indicative of a bioterror attack.

2. Unusual distribution of casualties—a high number of persons afflicted with respiratory illness, or a casualty distribution that is related to wind direction, might indicate that an aerosol attack has occurred. This could be corroborated by evidence of lower casualty rates among those working indoors, and in particular in areas with filtered air or closed ventilation systems.

3. Unusual distribution—a large number of rapidly fatal cases, with few recognizable signs and symptoms, may indicate exposure to multiple lethal doses of a biological agent from a single source.

4. Unusual disease pattern—the disease pattern associated with an attack utilizing a biological agent is likely to differ from those of a naturally occurring epidemic. For example, except for food borne outbreaks, disease incidence in naturally occurring epidemics usually increases over a period of weeks or months. However, in a bio-terrorism attack, the increase in disease incidence may be only hours or days. Furthermore, instead of the usual peaks and troughs evidenced in most natural outbreaks, a steady and increasing stream of patients will be seen in a bioterror attack, similar to a food poisoning outbreak.

5. Unusual disease outbreak—the recurrence of a vector-borne disease without the vector (e.g., a mosquito or a tick) or the occurrence of a disease that is highly unusual for the geographic area could indicate that a bio-terrorism attack has occurred.

6. Unusual disease symptoms—an unusually high prevalence of respiratory disease (e.g., pneumonia) from a disease that more often occurs naturally as a skin disease (e.g., inhalation in a natural outbreak) can be indicative of a bio-terrorism attack.

7. IIIness in animals and humans—an increased number of sick or dead animals, often of different species (e.g., rodents and dogs) at the same time as an increased number of human illness can be indicative of a bio-terrorism attack.

Where a biological attack has occurred at a site such as a mail facility, those tasked with the remediation effort typically need to address remediation for exposed goods (e.g., mail items), for vehicles utilized in the response effort, for facilities where an attack has (or may have) occurred, and for persons (including their personal gear) who are involved in the remediation effort.

For goods of relatively small size, such as mail items, exposure to accelerated electron beam irradiation with sufficient intensity and for a sufficient period of time can kill anthrax spores and thus decontaminate the goods. (Such treatment is widely used for sterilizing medical instruments, cosmetics, and pharmaceuticals—in a non-bio-terrorism context.) Testing has shown that such treatment does not produce or store any radiation in the treated goods. Moreover, it appears to produce no adverse health effects.

Turning particularly to a discussion of mail items suspected of contamination, the mail items may be arranged in a manner that will both ease their transportation (where this is necessary) and, more importantly, may optimize the efficiency of the irradiation treatment. First, mail items may be segregated both by size and based on the contents thereof. Letter-size mail items, i.e., those having a height of approximately four inches or less, a length of no more than approximately 11 and ½ inches, and a thickness of no more than approximately ¼ inch, may be optimally processed as hereinafter described. However, of these mail items, letter-size mail items containing plastics, credit-cards, compact disks and the like may be separated for alternative processing, because the method described herein has been shown to be harmful to such items at certain dosage levels.

The mail items may be positioned in a letter tray having a height of four inches. The mail items should be placed horizontally (i.e., flat) within the tray, and can be stacked to the height of the tray walls. However, care should be taken so that mail items are not positioned above the height of the walls.

The filled tray may be placed into a letter tray sleeve. The letter tray sleeve may be closed by taping it along its length. The purpose of the letter tray sleeve is to maintain the letters in position within the tray.

The sleeve, with the filled tray therein, may be inserted into a polyvinyl bag having a thickness of between about 3 and about 6 millimeters. (Generally, the use of a bag will be desired at higher dosage levels.) If used, the bag may be tied by goosenecking the opening and sealing it with duct tape. This sealed bag may be inserted gooseneck-end first into a second polyvinyl bag, also having a thickness of between about 3 and about 6 millimeters. The second bag may be sealed in like manner to the first—i.e., by goosenecking the end and sealing it with duct tape.

The double-bagged letter tray may be inserted into a cardboard box of sufficient dimension to receive the bagged tray, for purposes of securing it for travel. For a standard letter tray, the desired box dimensions may be as follows: (a) interior dimensions: 12 inches wide, 26 inches long, and 6.75 inches deep; (b) exterior dimensions: 12.25 inches wide, 26.5 inches long, and 7 inches deep. The dimensions may be varied as desired. The box may be sealed by taping it shut with packing-type tape, for example, tape having a width of about 2 to 3 inches. Duct tape may not be preferred far box-sealing purposes. The packing tape may be placed entirely around the width of the box.

Where the irradiation equipment is located remote from the facility where the mail has been packaged, the sealed boxes may be loaded onto vehicles for transport to the treatment location.

At the treatment location, the sealed boxes may be exposed to accelerated electron beam irradiation for a time period that is between approximately 30 minutes and approximately 1 hour. This amount of time has been shown to be optimal for decontamination purposes.

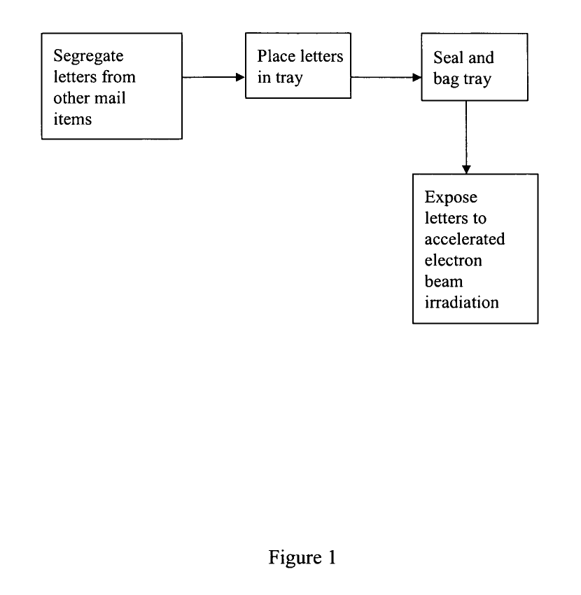

The general steps involved in the remediation of mail items suspected of possible exposure to a biological attack are depicted in the flow chart of FIG. 1. It should be recognized that some departure from the specific steps described herein, may be possible without departing from the spirit or scope of the present invention. Thus, it may be possible, for example, to substitute one thicker bag for the two described herein, or to use more than two bags, to eliminate the tray sleeve or to provide more than one tray sleeve.

Where a vehicle has been used to transport mail items (or other goods) that are at least suspected of possible contamination as a result of a bio-terrorism attack, it may be necessary to decontaminate the vehicle interior. For this process, a suitable decontamination area may be created. The features of such an area may include an underlying, impermeable layer that is strong enough to withstand the traction of tires. An additional feature may be a barrier, such as a berm, to prevent the run-off of liquids that may be used in the decontamination process and to allow their collection. Run-off prevention may be accomplished in other ways, for example, by providing an area with a sloped floor leading to one or more drain openings. Commercially available household bleach solution containing 5.25's hypochlorite, when diluted 10:1 is effective in routine decontamination of surfaces and instruments after working with B. anthracis.

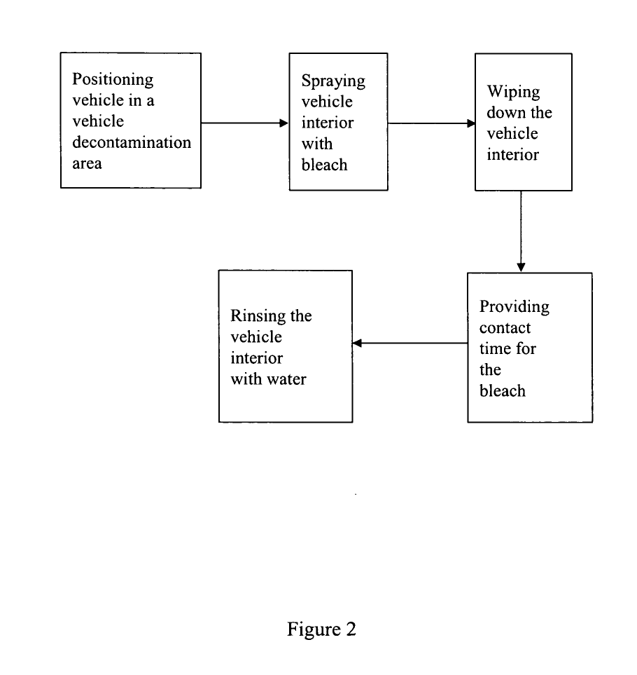

The vehicle is preferably positioned within a suitable decontamination area. The vehicle's interior may be sprayed using a decontamination solution. The vehicle may be permitted to stand exposed to the decontamination solution for a period of time (e.g., one hour) to allow sufficient contact time for the decontamination solution (e.g., residual detergent) to act. After the period of time, the vehicle interior may be wiped down by hand using clean cloths. The vehicle interiors may be thoroughly rinsed with clean tap water and allowed to air dry. The general steps associated with vehicle decontamination are illustrated in the flow chart of FIG. 2.

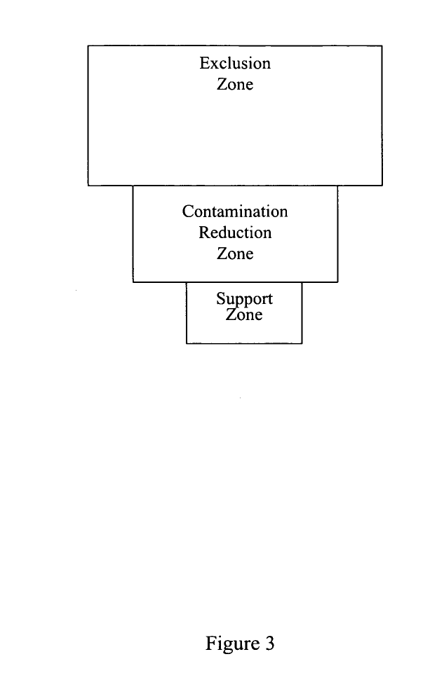

In order to undertake appropriate decontamination efforts with respect to a facility where a bio-terrorism may have occurred, a suspected contamination site and its surrounding areas is preferably divided into zones, including: (a) an exclusion zone—this is the area that includes the source of the incident and which is therefore contaminated or is at least suspected of having been contaminated; (b) a contamination reduction zone—this is the area adjacent to the exclusion zone and is utilized for the decontamination and rinsing of personal protective equipment (PPE); and (c) a support zone—these are located at the entry points to the contamination reduction zone. The position of the zones relative to one another—with the contamination reduction zone interposed between the support zone and the exclusion zone—is illustrated in the block diagram of FIG. 3.

The exclusion zone includes the area where an attack is suspected to have occurred and where decontamination efforts may be undertaken. Boundaries of the exclusion zone may be determined from an initial post-attack assessment and may be based on an identification of hazards. Entry to the exclusion zone occurs through the contamination reduction zone and should be permitted to persons wearing appropriate PPE's. The exclusion zone may be marked with appropriate signage in order to avoid accidental entry into the exclusion zone without proper equipment.

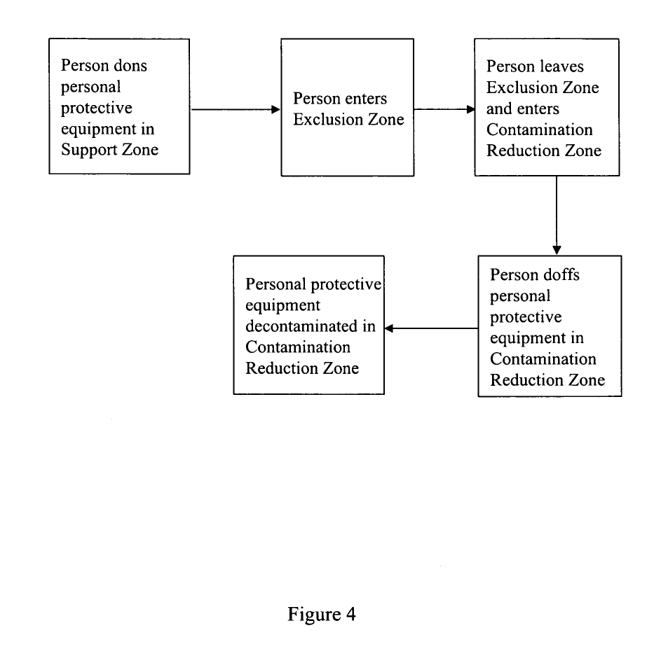

As noted above, the contamination reduction zone is adjacent to the exclusion zone and serves as a transition zone between the exclusion zone and the support zone. A person wearing a PPE (which has been donned in the support zone, discussed below) may pass through the contamination reduction zone and enter the exclusion zone. Such entrance may follow an inspection, which may occur in the support zone or in the contamination reduction zone, to ensure that the PPE is being worn correctly. The contamination reduction zone may also have a decontamination facility to be utilized by persons exiting the exclusion zone, and may be configured to permit the decontamination of PPE's. All PPE's should be removed before a worker exits the contamination reduction zone for the support zone. This method—donning of PPE in the support zone, entrance to the exclusion zone, exiting of the exclusion zone and passage into the contamination reduction zone, and decontamination of the PPE in the contamination reduction zone—is shown in the flow chart of FIG. 4.

The decontamination facility within the contamination reduction zone may include an enclosed tent system, having multiple stages (e.g., four). Within the tent system, and as more fully described below, decontamination, rinsing, and donning of PPE's will occur.

The support zone is a clean zone, and may be a temporary structure, such as a trailer, that is not a fixed part of a facility containing a contamination site. In this manner, the support zone can be pre-configured in advance of a bio-terrorism attack, and can be relatively quickly transported to the site and deployed for use. The support zone may contain equipment, a command post, and back-up personnel.

For persons entering the contamination reduction zone and thereafter the exclusion zone, the support zone is used for donning PPE's. For those exiting the contamination reduction zone, the support zone may be used for heat stress monitoring and showering. The support zone may also be used, for storing respirators and charging respirator power packs.

Persons exiting the exclusion zone and entering the contamination reduction zone subject their PPE's to a decontamination process. The purpose of this process is to prevent exposure to the persons wearing the PPE's and to prevent the spread of contamination to clean areas of the site.

With respect to the PPE, two layers of protective clothing may be worn. For example, it may be desired to utilize an outer suit that is a polycoated Tyvek type and an inner suit that is a standard Tyvek type. Or, it may be desired to provide a standard Tyvek type suit for the outer layer with scrubs underneath. Where heat stress is not a concern, an outer Saranex Tyvek type suit may be worn over a polycoated Tyvek type suit.

In addition to the PPE, a worker entering an exclusion zone may be wearing other equipment (e.g., gloves, booties, safety goggles, and a respirator). The respirator may be a powered air purifying respirator, equipped with a suitable cartridge, such as an organic vapor/acid gas cartridge. Cartridges may be discarded after each exit from the contamination reduction zone and randomly sampled for evidence of contamination. The gloves may be nitrile and the worker may wear two pairs—an inner pair and an outer pair. The worker may also wear two inner pairs and an outer pair. For certain activities, an outer glove comprised of leather or the like may be desired, also, for certain activities, work boots, such as those having steel toes, may be desired. Tape may be used to seal the sleeve and pant leg openings. In some instances, the worker may wear a hood, which may be taped to the safety goggles.

As noted briefly above, the contamination reduction zone may be divided into four stages or areas, with different decontamination activities to occur in each area. Each stage or area should be defined by a separate tent, within the contamination reduction zone, and a person passes from area to area by exiting one tent and passing into another tent. Some variation in the number of areas, and in the particular activities to occur in each, is possible, and may be preferred depending on the circumstances.

In an embodiment consistent with the present invention, de-contamination may proceed as follows:

### Area 1

The PPE is rinsed with a soap solution, with the rinse emptying into a bleach type solution. Rinsing may be conducted twice. The front and back of suits may be rinsed and the process may be repeated. If the worker was wearing a hood, this is rinsed as well. If not, the worker should lightly spray his or her head with the soap solution. The PPE may be rinsed with water.

The tape may be removed from the sleeve and pant leg openings. The worker may also remove the PPE and the outer and the first pair of inner gloves. The PPE may be placed in a container and sprayed with a bleach solution, with the solution left thereon for approximately one hour contact time.

### Area 2

In the second area, the worker may remove his or her inner scrubs by cutting and/or tearing them away. These should not be pulled over the head. The booties are also removed. The discarded scrubs may be placed in a container and sprayed with a bleach solution, with the solution left thereon for approximately one hour contact time. The discarded booties may be placed in a container with the scrubs, or may be placed in a separate container arid sprayed with a bleach solution, with the solution left thereon for approximately one hour contact time.

### Area 3

In this area, the worker removes his or her respirator (or other respiratory protection), as well as the second pair of inner gloves. The inner gloves may be placed in a container and sprayed with a bleach solution, with the solution left thereon for approximately one hour contact time.

The respirator may be placed into a bleach solution for approximately one hour. Its parts should be wiped down with the bleach solution, with the solution left thereon for an approximately one hour contact time. Thereafter, the respiratory should be rinsed with water and allowed to air dry.

### Area 4

Area 4 may include a shower area. All workers may be required to shower before leaving the contamination reduction zone. Showering should be with soap and water.

In an embodiment consistent with the present invention, de-contamination may proceed as follows:

### Area 1

The PPE may be rinsed with a soap solution, with the rinse emptying, into a bleach type solution. Rinsing may be conducted twice. The front and back of the suits may be rinsed and the process may be repeated. If the worker was wearing a hood, this may be rinsed as well. If not, the worker may lightly spray his or her head with the soap solution.

### Area 2

In this area, the PPE may be rinsed with water. The tape may removed from the sleeve and pant leg openings. The worker may also removes the PPE and the outer and the first pair of inner gloves. The PPE may be placed in a container and sprayed with a bleach solution, with the solution left thereon for approximately one hour contact time.

### Area 3

In the third area, the worker may remove his or her inner scrubs by cutting and/or tearing them away. These should not be pulled over the head. The booties may also be removed. The discarded scrubs should be placed in a container and sprayed with a bleach solution, with the solution left thereon for approximately one hour contact time. The discarded booties may be placed in a container with the scrubs, or may be placed in a separate container and sprayed with a bleach solution, with the solution left thereon for approximately one hour contact time.

The worker may remove his or her respirator (or other respiratory protection), as well as the second pair of inner gloves. The inner gloves are placed in a container and sprayed with a bleach solution, with the solution left thereon for approximately one hour contact time.

The respirator may be placed into a bleach solution for 15 minutes. Its parts may be wiped down with the bleach solution, with the solution left thereon for an approximately forty-five minute contact time. Thereafter, the respiratory may be rinsed with water.

### Area 4

The fourth area may include a shower area. All workers may be required to shower before leaving the contamination reduction zone. Showering may be with soap and water.

It is noted that adverse weather conditions can impair the decontamination process. For example, severe winds can damage structures positioned around a building to aid in the decontamination effort. This may include materials used to seal a building to be decontaminated, structures related to the generation and delivery of a decontaminant, and the tent/trailer structures described herein. In addition, severe winds can cause an object to become airborne, creating what is known as a “missile hazard,” creating a danger to workers and to structures on the site.

Accordingly, weather conditions may be monitored, so that adequate warning can be provided of the onset of adverse conditions, such as hurricane-force winds, tornados, lightning, heavy rain, snow, ice, flooding, and severe storms. Preferably, wind speed, wind direction, ambient outside temperature, ambient relative humidity, and barometric pressure are monitored.

Monitoring should be conducted by a duly designated person, such as a site health and safety officer (“SHSO”). The SHSO may report information received as a result of such monitoring to an appropriate person (such as an incident commander) so that appropriate action may be taken. The incident commander may be, for example, a site superintendent, site manager, and/or site health and safety director.

A warning system may be used, based on, at least in part, the likely incidence of destructive winds. Destructive winds may be considered to be winds that are determined to have the potential to cause property damage or personal injury. Generally, these are winds that reach or exceed the force of a tropical storm; i.e., having a speed of greater than 34 knots.) Multiple conditions of readiness may be created based on the possible arrival time of such winds. For example, five conditions of readiness are characterized below:

Condition V—destructive winds are possible at the site within 96 hours. This condition continues until the storm or condition that may produce destructive winds is downgraded or changes track so as to no longer pose a threat to the site, or until Condition IV begins.

Condition IV—destructive winds are possible at the site within 72 hours. This condition continues until the storm or condition that may produce destructive winds is downgraded or changes track so as to no longer pose a threat to the site, or until Condition III begins.

Condition III—destructive winds are possible at the site within 48 hours. This condition continues until the storm or condition that may produce destructive winds is downgraded or changes track so as to no longer pose a threat to the site, or until Condition II begins.

Condition II—destructive winds are possible at the site within 24 hours. This condition continues until the storm or condition that may produce destructive winds is downgraded or changes track so as to no longer pose a threat to the site, or until Condition I begins.

Condition I—destructive winds are possible at the site within 12 hours.

It should be noted that the accuracy of predicting where, for example, a hurricane landfall will occur is very low, more than 24 hours in advance of a storm. 72 hours before landfall, the maximum probability of hurricane landfall location is 10%; 48 hours before landfall, the maximum probability is 13-18 percent; 36 hours before landfall, the maximum probability is 20-25 percent; 24 hours before landfall, the maximum probability is 35-45 percent; and 12 hours before landfall, the maximum probability is 60-70 percent. As a result, it may be desired to provide fewer than five conditions of readiness, or perhaps more than five, with the time interval from landfall defining each condition of readiness to be varied as desired.

Corresponding to the conditions of readiness may be activities at the decontamination site to prepare for the possible onset of destructive winds. The following are examples:

Condition V—conduct normal daily site cleanup and maintain good housekeeping practices.

Condition IV—conduct normal daily site cleanup and maintain good housekeeping practices. Collect and store in piles or containers scrap lumber, waste material, and rubbish for removal and disposal at the end of each workday. Other objects that could become missile hazards should also be removed.

Condition III—Condition. IV requirements should be maintained. In addition, personnel should begin securing the site and taking those actions necessary for Condition I that take more than 18 hours to complete. Any routine activities that interfere with securing operations should be ceased.

Condition II—routine activities should be curtailed or ceased entirely until securing operations are complete. Machinery, tools, equipment and materials should be secured or removed from the site. Any remaining missile hazards should be secured or removed from the site.

Condition I—complete all remaining securing actions, secure the site, and evacuate.

Progress of the potential destructive wind source may be monitored regularly, in order to determine whether a particular condition is applicable or whether a change in condition status is appropriate. The frequency of such monitoring should increase as progress in the direction of Condition I continues. For example, in Conditions V, IV, and III, monitoring the progress and location of the potential destructive wind source may occur three times per day. In Condition II, such monitoring should occur about every three hours. In Condition I, such monitoring should be continuous.

Destructive winds are not the only weather hazard that may be of concern to a de-contamination site. For example, lightning also poses a potential hazard. When a lightning flash is observed in the immediate area or within a range of about 4 miles, outside activities should be suspended. Lightning distance may be measured using the flash/bang technique, according to which lightning is one mile away for each five seconds that passes from the time of observing the lightning flash to hearing the associated thunder. Indoor activities may be continued, except for the use of electrical equipment, telephones and computers. Outdoor activities may be resumed when the lightning moves beyond 4 miles from the site.

Fumigation may also be used in a process to decontaminate a facility. The temperature and humidity of the facility are preferably controlled to a desired level. ClO₂ s a preferred fumigation agent. ClO₂ may be introduced through the HVAC system of the facility. Sampling of different areas and items within a facility may aid in determining the extent of contamination of the facility.

Purposes for sampling and analysis activities conducted on a facility include characterizing the extent of contamination within a facility. After the initial characterization of contamination, sampling activities may be continued to determine the full extent of contamination due to cross-contamination by equipment, personnel, and air to confirm successful decontamination activities within the facility and on items removed from the facility.

Sampling methods may include using HEPA vacuum samples and wipe samples. In addition to these types, a combination wipe and swab sample (WOB) and air samples may be obtained periodically. Samples may be evaluated using a polymerase chain reaction (PCR) methodology.

When samples are reported positive, a spore concentration may be estimated for the sample. The concentrations may be utilized to estimate the overall spore concentration in the facility. The concentration may aid in giving an estimate of the level of spore contamination left in the facility.

As noted above, samples may be taken to evaluate a location and an amount of contamination within the facility. Other areas may be identified depending on the likelihood of cross-contamination. Additional samples may be taken in different areas to better understand facility and mail pathways. For example, areas of concern may include areas that may be contaminated to airflow in the facility. The samples may be biased towards the unsealed doors.

Mail processing equipment handling contaminated mail in the facility may be sampled. Machines known to be contaminated may be sampled initially and most intensely. Samples may be taken on surfaces having contact with mail, primarily horizontal surfaces, and porous items on the machines. The other mail processing machines may be sampled because the spores may become airborne and therefore spores may have a potential to move anywhere.

Another area of focus may be areas of human contact with contaminated mail (e.g., letter cases). Letter cases used in the Government Mail Section contain individual slots in which the mail is hand sorted for distribution. Sampling on the letter cases may be biased to areas that came into contact with mail and also had a moderate to high probability of human contact.

Sampling may be conducted in other areas of the facility including offices, community areas, galleries, walls, floors, and ceilings. Samples may be biased towards areas of moderate to high human contact. In the offices, samples may be taken on the floor in the entrance of the office, desks, and computer equipment. Community area samples may include the break rooms, restrooms, and locker rooms. Samples may also be taken in transition areas from the tile flooring to carpeted areas. Ceiling samples may be taken on skylights, vents, trusses, and supports (e.g., above the machines).

Based on historical sampling events throughout the facility, regions within the facility may be ranked. The ranking may be high, medium or low. A region ranked high indicates a high probability of contamination (e.g., B. anthracis spores being present). These areas may have the largest number of historical positive samples with the highest spore counts. The regions that are ranked as medium may have some historical positive samples and all areas with positive samples have been decontaminated. The regions ranked low are those zones with few if any historical positive samples, and those regions with positive samples which were decontaminated.

There may be several types of sampling that may be performed to determine contamination. Confirmation sampling may be performed to verify, for example, that either there was no contamination on an item that could not be decontaminated (i.e. files, computer hard drives) or that the area or item was successfully decontaminated.

Confirmation samples may be taken in perimeter areas to ensure that on-site employees may work in those areas without risk of contamination. These areas may include the facility's roof and tables used to process mail. Decontamination tents and rental equipment being returned may be sampled to ensure no cross-contamination.

Decontamination may not be successful for some line items. For example, computers, files, pictures, Postal Inspectors' items, etc, in which the decontamination procedures may ruin the materials that needed to come out. The sampling for these materials is done as needed, and 100 percent of the surface areas of the materials being brought out should be sampled.

In addition, wastewater and rodent carcasses may be sampled as necessary. The wastewater may be sampled prior to disposal to ensure that the water was not contaminated with spores. Rodent traps may be set up around the exterior and the interior of the facility to minimize the potential movement of contaminated rodents within a community. Any rodents caught may be sampled as possible utilizing a HEPA vacuum.

Further, regions in the facility having positive sample results may undergo spot decontamination with HEPA vacuuming and/or anti-microbial solution. These areas may be re-sampled to verify effective decontamination.

Clearance sampling may concern samples taken on items to be released (e.g., for use in the U.S. Postal Service, on the job site, or to be returned to its owner). There may be two different purposes for the samples being taken. For example, rental equipment being taken off-site may be considered a clearance sampling, but if the equipment were to be serviced on site, the samples may be considered as confirmation samples.

Examples of items considered for clearance sampling include postal vehicles and trailers, decontaminated rolling stock from inside the facility, and mail tubs and mail bins, stacked on pallets and shrink-wrapped. Miscellaneous items included trashcans, hand trucks, and signage from the facility.

Sampling may also occur after fumigation of the facility. After successful fumigation (as explained below), based on data collected during fumigation and from analysis of biological indicators, the facility may be tentatively regarded as “clean.” The facility may not be treated operationally as cleared of contamination (e.g., by B. anthracis threat) until environmental samples are collected, tested, and that no viable B. anthracis spores are detected. The post-fumigation environmental sampling may be designed to detect elevated risk of contracting some form of an anthrax infection by working in the facility.

If a criterion of “no live spores” is given to a contaminated area, any detection of B. anthracis spores in confirmation samples may be unacceptable. The most that may be done to directly verify that there are no live spores is to obtain a large number of samples and using sensitive sampling and analytical methods, find no detectable evidence of contamination.

If the fumigation provides sporicidal efficacy, an occurrence of a B. anthracis colony-forming unit (CFU) on a facility surface or in facility air will be an exceedingly rare event. CFU counts per sample are not directly observed. The observations may include non-detection sample results and estimated CFU counts for the positive detection samples. After fumigation, estimated CFU counts for positive detection samples may be quite low. Therefore, information available about B. anthracis concentration may be in the detect rate. Therefore, an appropriate test statistic may be a number of detects. A “no growth” standard may indicate that an acceptable number of detects is zero.

For example, a statistical calculation may be done to determine the probability that an individual would contract an anthrax infection. If it is estimated that the calculation yields 1×105 over the course of a year based on the acceptable occupational cancer risk, the probability translates to approximately 2.5×106 for a 90-day period. The risk of contracting inhalation anthrax may be based on an assumption that one spore could cause an infection. The risk of contracting cutaneous anthrax was based on the assumption that ten spores would cause infection. The amount of time employees spent in the facility may be considered as well as an estimate of number of spores remaining in the facility before fumigation efforts began. Other factors considered in the calculations may include the United States Environmental Protection Agency (USEPA) published recommended average inhalation rates and an estimate of surface area with which an employee will be in direct contact.

With regard to biological indicators, there may be two types of spore strips that may be utilized in the facility during fumigation: spore strips that have a specific log of Bacillus subtilis (B. subtilis) indicator spores and steri-charts that have a series of known concentrations of B. subtilis spores. The strain of bacterium to be used in the strips during fumigation may be B. subtilis. This strain is most often used to determine the effectiveness of ethylene oxide sterilization and has been used in pharmaceutical applications for the past 15 years to demonstrate effective treatment with ClO₂. The log of a spore strip may indicate a minimum kill probability locally at the strip if the strip shows no growth after culturing. Both spore strips and steri-charts may be used in the facility to assess the effectiveness of fumigation conditions in the facility. The spore strips may be used to provide data that demonstrates that the ClO₂ has reached sufficient concentration, with proper conditions of temperature and relative humidity, to kill the B. subtilis spores on the spore strips. Therefore, by implication, this may also kill the B. anthracis spores in the facility.

For example, placement of the spore strips in the facility will be such that two sequential ClO₂ treatments may be measured. Therefore, two sets of each setup may be placed at each sample location. Five percent of the sample locations may also have a duplicate set of strips placed, and ten percent of the sample locations will have both spore strips and steri-charts.

Steri-charts may also be attached to the center of the ClO₂ gas sampling spider. The gas sampling spiders may be a composite sampling apparatus with either six or twelve sampling lines per sample. An adequate number of composite gas sampling locations may be provided with which to directly correlate dose clock with kill probability as measured by statistical analysis of the steri-chart results.

With regard to environmental sampling, the number of samples proposed for post-fumigation clearance may be a predetermined number of environmental samples and locations of spore strips (e.g. 3,962 environmental samples and 3,513 locations for spore strips). This frequency may be based on the following guidelines provided by the regulatory agencies including the Centers for Disease Control and Prevention (CDC), USEPA, OSHA and the Armed Forces Radiobiology Research Institute (AFRRI): 1 sample or spore strip per 300 square feet (ft²) in random locations; 1 sample or spore strip per 100 ft² in biased locations; and 1 sample or spore strip per 100 ft² in focused locations. The locations of the samples are determined using three major criteria. This may maximize the areas sampled, and thereby minimize the likelihood that spores in the facility will go undetected.

The first major group of sample locations may be random samples based strictly on the spatial square foot dimension of the facility. These locations may be designated using software (VSAMPLE) to maximize the location points.

The second type of samples may be biased locations. These sample locations may be biased to place spore strips in poor gas, humidity or temperature conditions and the environmental samples may be placed where there is a higher likelihood of contamination. These locations may based on judgment and general knowledge of the use of the facility.

Third, sample locations may be designated based on historically contaminated locations, these sample locations may be denoted as “focused” sample locations. These location points may also be locations where B. anthracis spores have been found in the past.

Several options may be available to fumigate the facility for B. anthracis spores including ozone, hydrogen peroxide, and para-formaldehyde. Each of these agents may have potential risks to employees and the community during the decontamination, residual risks to the employees, or risks to the facility.

Distribution of ClO₂, may be considered to be the safest method. The fumigation and treatment system will provide effective remediation of the B. anthracis spores for the facility and thereby protect human health and the environment. Although only ClO₂ is discussed in detail below, it is understood by those skilled in the art that systems and methods consistent with the present invention may include fumigation by any gaseous agent suitable for fumigation.

Due to the nature of ClO₂, actions may be implemented to the facility to ensure that the gas would fumigate properly. ClO₂ breaks down readily in ultraviolet light, so all exterior windows, glass doors, and skylights may require covering from outside the facility with light-blocking materials. All non-essential lighting inside the facility may be turned off and immediately prior to fumigation, the remaining lights in the facility will be shut off to eliminate any artificial light sources in the facility.

Because ClO₂ is a toxic chemical, precautions may be taken to minimize leakage from the facility. All accessible points of air infiltration-exfiltration may be sealed, including exterior doors, windows, and the skylights. All heating, ventilation, and air conditioning (HVAC) units may be sealed including exhaust flues, vents, and fans that may be located within the facility. All cracks, expansion joints, electrical conduits, pipe penetrations, and seams in the facility exterior may also be sealed. Gaps between the roof decking and the block envelope wall may be sealed from inside the facility.

Other measures may be taken to further prepare the facility for fumigation. Various materials may be removed from the facility prior to fumigation including those items that would readily absorb ClO₂. These items may include: several types of mail, packages, rolling stock, plastic mail bins, paper files and binders, garbage and trash, debris and cardboard, canvas bags, clothing and personal effects, stamps, money, ammunition and weapons, biohazard boxes, furniture and chairs, chemical containers, electrical appliances, evidence, ceiling tiles, food stuffs, and vending machine contents. These items may be decontaminated and sent offsite for incineration or as applicable, re-entry into a system of equipment sharing between facilities.

The HVAC systems may undergo preparation for the fumigation including repairs and maintenance on existing air handling units (AHUs). Filters in AHUs may be removed, decontaminated with an anti-microbial solution, double-bagged, sealed, and sent offsite for incineration. Insulation may be removed from inside the AHU cages and housing for disposal. Outside air damper arms may be physically disconnected so that louvers would not respond to a call for outside air. Boilers for the facility may be isolated by construction of a decontamination room around them and ducted to allow the combustion air intake and exhaust vents to function normally. Additionally, an upgraded software and hardware computer package may be installed to allow remote operating control of the HVAC systems from a station located outside the facility.

In addition, other factors that may potentially hinder the penetration of ClO₂ gas during fumigation may be considered. For example, items such as doors, refrigerators, vaults, desk drawers, microwave ovens, lockers, filing cabinets, and supply and storage cabinets were opened as possible to facilitate the gas dispersion. Additionally, toilets throughout the facility may be sealed to prevent sewer gas from entering the facility under negative pressure.

Engineering design of the facility fumigation treatment systems may include providing redundancy of critical components, process safety interlocks, and examining component suitability for the intended service conditions. Modeling, pilot-scale, and bench-scale testing may be performed to support the technical design. Large-scale testing may be performed to demonstrate the safe and effective operation of the as-built systems. Collectively these engineering design efforts may minimize the risk of equipment failures or poor performance during fumigation while maximizing operational safety for the workers and public.

For example, redundant or intentionally over-designed capacity may be provided in many subsystems including: 100% redundant electric power generation capacity, 100% redundant negative air drafting capacity, 100% redundant chemical pumping capacity, 25% excess liquid pumping capacity, excess sensible heat capacity, excess humidification capacity, excess dehumidification capacity, excess chilling capacity, and excess liquid waste storage capacity.

Process safety interlocks may be provided to assure that certain unsafe or undesirable operations cannot be inadvertently performed. For example, sump pumps inside the facility run off level controllers to prevent tank overflowing. In addition, induction draft fans automatically shut down at too great a negative pressure inside facility, safety relief valves automatically open to prevent too great a negative pressure inside facility, draft fans automatically control negative pressure from the five most positive transducers, and re-heaters shut off if air flow from facility becomes too low.

Hardware and instrument components may be selected to provide suitable service under the anticipated conditions of temperature, humidity, and potentially corrosive gas exposure. For example, temperature-relative humidity sensors and the HEPA filters may be tested by challenging them through exposure to ClO₂ gas during the design process. Pilot tests may be performed to assure that various mechanical components and instruments function as intended. These pilot tests include gas emitter stripping efficiency, gas emitter scrubbing efficiency, wet chemical scrubber tower efficiency, mixing and transfer fan effectiveness, activated carbon bed effectiveness, and gas sample tubing effectiveness.

It should be recognized that the individual methods described herein, including remediation of goods (e.g., mail items), vehicles, facilities, and personal protective equipment could be utilized individually or as part of a comprehensive response to a biological attack. Moreover, while the invention has been particularly shown and described with reference to embodiments thereof, it will be understood by those skilled in the art that the foregoing and other changes in form and details may be made therein without departing from the spirit and scope of the invention.

For example, additional packaging steps may be desired for mail items, or certain ones may be modified or eliminated, while still preserving the essential effectiveness of the irradiation treatment. Some departure from the contact times described herein may be permitted without unduly reducing the effectiveness of the bleach solution treatment. The concentration of the bleach solution may be altered slightly, to make it slightly more or slightly less concentrated, while maintaining decontamination effectiveness and compliance with applicable health arid safety guidelines. It may be desired to wear additional personal protective equipment, beyond that described herein, which additional equipment would also need to be remediated. The contamination reduction zone could have more, or fewer, decontamination areas than are described herein. The types of weather conditions monitored; the number, duration, and qualifying factors for conditions of readiness; and the types of permitted/recommended activities for particular conditions of readiness can also be varied.

1. A method for decontaminating a facility, said method comprising:
establishing an exclusion zone having a suspected contaminant in one or more areas;
establishing a support zone;
establishing a contamination reduction zone between the exclusion zone and the support zone such that the support zone is located at an entry point of the contamination reduction zone;
creating a pressurized environment inside the exclusion zone;
introducing an agent to the pressurized environment in order to substantially eliminate the contaminant; and
sampling at least one area in order to determine a presence of the contaminant in a sample.

2. The method of claim 1, wherein the contaminant comprises the bacterium Bacillus anthracis.

3. The method of claim 2, further comprising removing items from the facility that are susceptible to damage by the agent.

4. The method of claim 2, wherein the pressurized environment is a negative pressure environment.

5. The method of claim 2, wherein the agent is ClO₂.

6. The method of claim 2, wherein said sampling includes using at least one of HEPA vacuum samples and wipe samples.

7. The method of claim 2, wherein said sampling includes using a polymerase chain reaction methodology.

8. The method of claim 2, further comprising estimating a spore concentration of the facility from a spore concentration of the sample.

9. The method of claim 8, further comprising ranking one or more regions in the facility based upon a spore concentration of a sample taken from the one or more areas.

10. The method of claim 2, wherein the sampling includes at least one of confirmation sampling and clearance sampling.

11. The method of claim 1, further comprising dividing the exclusion zone into regions.

12. The method of claim 11, further comprising ranking the zones based upon a spore concentration of a sample taken from each zone.

13. The method of claim 1, wherein the agent is introduced through the facility HVAC system.

14. The method of claim 5, wherein the agent is introduced through the facility HVAC system.

15. The method of claim 1, wherein the contamination reduction zone includes a decontamination facility for decontaminating personal protective equipment.

16. The method of claim 1, wherein the decontamination facility is a tent with a plurality of stages for decontamination of personal protective equipment.
